(19) United States
(12) Patent Application Publication
(10) Pub. No.: US 2022/0380806 A1
(43) Pub. Date: Dec. 1, 2022

(54) GENE THERAPY FOR THE REGENERATION OF AUDITORY HAIR CELLS

(71) Applicant: ICAHN SCHOOL OF MEDICINE AT MOUNT SINAI, New York, NY (US)

(72) Inventors: Pin-Xian Xu, New York, NY (US); Jun Li, New York, NY (US); Jinshu Xu, New York, NY (US)

(21) Appl. No.: 17/773,675

(22) PCT Filed: Nov. 25, 2020
(86) PCT No.: PCT/US2020/062178

§ 371 Date: May 2, 2022

Related U.S. Application Data
(60) Provisional application No. 62/941,112, filed on Nov. 27, 2019.

(51) Int. Cl. C12N 15/86; C07K 14/47; A61P 27/16

(52) U.S. Cl. CPC C12N 15/86; C07K 14/4705; C07K 14/4703; A61P 27/16; C12N 2750/14143; A61K 48/00

(57) ABSTRACT

Provided herein are compositions and methods for treating and preventing hearing loss, for treating and preventing a disorder associated with loss, damage, or absence of sensory auditory hair cells, and/or for improving auditory function in a subject in need thereof. Also provided are compositions and methods for the generation of auditory hair cells that allow perception of stimuli in a subject in need thereof.

### CROSS-REFERENCE TO RELATED APPLICATION

This application claims priority to U.S. Application No. 62/941,112, filed Nov. 27, 2019, which is incorporated herein by reference it its entirety.

### STATEMENT REGARDING FEDERALLY SPONSORED RESEARCH

This invention was made with government support under RO1DC014718 awarded by the National Institutes of Health. The government has certain rights in the invention.

### REFERENCE TO SEQUENCE LISTING

The official copy of the Sequence Listing is submitted concurrently with the specification as an ASCII formatted text file via EFS-Web, with a file name of “084284-00210 sequence listing_ST25.txt”, a creation date of Nov. 24, 2020 and a size of 17.3 kb. The Sequence Listing filed via EFS-Web is part of the specification and incorporated in its entirety by reference herein.

### FIELD OF THE INVENTION

The present invention relates generally to the field of molecular biology and medicine. More particularly, the methods and compositions herein are useful for treating hearing loss associated with the loss, damage, or absence of auditory hair cells.

### BACKGROUND

In the United States, almost thirty million people exhibit some level of deafness or hearing loss. While this disability is rarely life threatening, it impairs more people psychologically and economically than epilepsy, multiple sclerosis, spinal injury, stroke, and Huntington's and Parkinson's diseases combined.

The vast majority of the cases of hearing impairment are caused by a loss of auditory hair cells, the sensory cells in the inner ear that transduce sounds into neural signals. This type of hearing loss is called “sensorineural hearing loss” and can be caused by a number of factors including sound trauma, ototoxic drug exposure, disease, viral infection, and genetic disorders. Severe to profound sensorineural hearing loss affects one in every thousand children born in the United States country, with half of these due to hereditary causes. In most cases of severe to profound hearing loss the cochlear nerve is still intact. For these individuals hearing function can often be restored with an electronic cochlear implant. However, due to the myriad of disadvantages associated with the expensive, low-fidelity, battery-powered, programmed external devices that are currently available for the hearing-impaired, methods that actually treat sensorineural hearing loss and thus enable people to regain their hearing without the use of electronic devices are urgently needed.

### SUMMARY OF THE INVENTION

The disclosure relates to methods and compositions for treating and preventing hearing loss, for treating and preventing a disorder associated with loss, damage, or absence of sensory auditory hair cells, and/or for improving auditory function in a subject in need thereof.

In one aspect, provided are one or more viral vectors comprising one or more polynucleotides encoding one or more gene products, wherein the gene products are selected from the group consisting of Six1, Eya1, Atoh1, Pou4f3, and Gfi1.

In some embodiments, the gene product is Six1.

In some embodiments, the gene products are Six1 and Eya1.

In some embodiments, the gene products are Six1, Atoh1, and Pou4f3.

In some embodiments, the gene products are Six1, Atoh1, Pou4f3, and Gfi1.

In some embodiments, the viral vector is an adenoviral (AV) vector, adeno-associated virus (AAV) vector, retroviral vector, lentiviral vector, or Herpes simplex type 1 (HSV1) vector. In some embodiments, the viral vector is an AV or AAV vector.

In some embodiments, the expression of the one or more gene products is inducible.

Also provided herein is a pharmaceutical composition comprising one or more vectors disclosed herein and pharmaceutically acceptable carrier.

In one aspect, provided is a method of treating damage to or loss of cochlear hair cells in a subject in need thereof, the method comprising:
(a) administering to a supporting cell in the organ of Corti in a subject in need thereof one or more vectors disclosed herein; or
(b) administering to the subject a cell transduced with one or more vectors disclosed herein.

In one aspect, provided is a method of generating cochlear hair cells in a subject in need thereof, the method comprising:
(a) administering to a supporting cell in the organ of Corti in a subject in need thereof one or more vectors disclosed herein; or
(b) administering to the subject a cell transduced with one or more vectors disclosed herein.

In one aspect, provided is a method of increasing hearing function in a subject in need thereof, the method comprising:
(a) administering to a supporting cell in the organ of Corti in a subject in need thereof one or more vectors disclosed herein; or
(b) administering to the subject a cell transduced with one or more vectors disclosed herein.

In some embodiments, the cell transduced with one or more vectors is a supporting cell.

In some embodiments, the subject has hearing loss. In some embodiments, the subject has age-related hearing loss, hereditary hearing loss, noise-induced hearing loss, disease-associated hearing loss, or hearing loss resulting from trauma.

In one aspect, provided is a viral vector for use in the treatment of hearing loss in a subject in need thereof, the viral vector comprising one or more polynucleotides encoding one or more gene products, wherein the gene products are selected from the group consisting of Six1, Eya1, Atoh1, Pou4f3, and Gfi1.

In one aspect, provided is a cell for use in the treatment of hearing loss in a subject in need thereof, wherein the cell is transduced with one or more vectors comprising one or more polynucleotides encoding one or more gene products, wherein the gene products are selected from the group consisting of Six1, Eya1, Atoh1, Pou4f3, and Gfi1

### BRIEF DESCRIPTION OF THE FIGURES

FIGS. 1A and 1B illustrate a Six1 gene therapy experiment. FIG. 1A shows the drug treatment schedule used in Example 2. FIG. 1B shows the structure of the organ of Corti. IHC=inner hair cells. OHC=outer hair cells. IBC=inner border cells. IPhC=inner phalangeal cells. PC=inner and outer pillar cells. DC=Deiters' cells. GER=great epithelial ridge. LER=lesser epithelial ridge. H=Hensen's cells.

FIGS. 2A, 2B, and 2C illustrate that Six1 gene therapy can be successfully be used for hair cell regeneration and hearing restoration. Anti-Myo7a (hair cells) or anti-Sox2 (supporting cells) staining or Cherry of P32 adult cochleae of R26^{FloxpSix1mCherry} treated with Saline (FIG. 2A), gentamicin (FIG. 2B) or Sox2^{CreER};R26^{FloxpSix1mCherry} treated with both gentamicin and tamoxifen (FIG. 2C). IHCs=inner hair cells; OHCs=outer hair cells.

### DETAILED DESCRIPTION OF THE INVENTION

There are six distinct sensory organs in the mammalian inner ear: the three cristae of the semicircular canals, the two maculae of the saccule and utricle, and the organ of Corti of the cochlea. The mammalian inner ear sensory organ for hearing—the organ of Corti—houses two types of auditory hair cells, inner hair cells (IHCs) and outer hair cells (OHCs), both of which are important for the detection and processing of auditory information. These hair cells are surrounded by supporting cells, a heterogeneous group of cells which are important for cochlear homeostasis. Supporting cells are comprised of one inner border, one inner phalangeal, inner and outer pillar, three rows of Deiters' cells and Henson's cells aligned in a medial-to-lateral direction. All supporting cell subtypes differentiate from common precursors.

Mature mammalian auditory hair cells are incapable of regeneration. Once damage has occurred in these cells, the degeneration process is often irreversible. Accordingly, failure to generate or maintain these epithelial cells in the organ of Corti causes irreversible deafness due to lack of regenerative capacity of the cochlea. In contrast, the less differentiated sensory epithelia within the inner ears of developing humans and non-mammals of all ages are capable of more significant hair cell regeneration after damage, and non-mammals can recover sensory function.

Accordingly, provided herein are compositions and methods of regenerating auditory hair cells by stimulating the formation of an auditory hair cell from a target cell.

Target Cells

Provided herein are compositions and methods for stimulating the formation of an auditory hair cell from a target cell. As used herein “target cell” and “target cells” refers to a cell or cells that are capable of undergoing conversion (e.g., differentiation) to or towards a cell or cells that have characteristics of auditory hair cells.

In one embodiment, the target cell is an inner ear supporting cell. Inner ear supporting cells (also referred to as supporting cells in the organ of Corti) include inner border, Deiters' cells, inner and outer pillar cells, inner phalangeal cells, and Hensen's cells. In one aspect, provided are compositions and methods for stimulating the formation of one or more auditory hair cells from one or more inner ear supporting cells. In one embodiment, the step of stimulating the formation of one or more auditory hair cells from one or more inner ear supporting cells includes the step of stimulating the inner ear supporting cells to enter the cell cycle, then stimulating at least some of the progeny of the inner ear supporting cells to differentiate to form one or more auditory hair cells. In one embodiment, the step of stimulating the formation of one or more auditory hair cells from one or more inner ear supporting cells includes the step of stimulating the de-differentiation of inner ear supporting cells to a multipotent state, then stimulating at least some of the multipotent cells to differentiate to form one or more auditory hair cells.

Additional target cells that can be used in the methods disclosed herein include, but are not limited to, e.g., stem cells (e.g., inner ear stem cells, adult stem cells, bone marrow derived stem cells, embryonic stem cells, mesenchymal stem cells, skin stem cells, and fat derived stem cells), and progenitor cells (e.g., inner ear progenitor cells). Prior to treatment with the compositions and methods described herein, each of these target cells can be identified using a defined set of one or more markers (e.g., cell surface markers) that is unique to the target cell. A different set of one or more markers (e.g., cell surface markers) can also be used to identify target cells that have experienced a partial or complete conversion (e.g., partial or complete differentiation) to or towards a cell that has characteristics of auditory hair cells. Target cells comprise or can be generated from stem cells isolated from a mammal, such as a mouse or human, and the cells can be embryonic stem cells or stem cells derived from mature (e.g., adult) tissue, such as the inner ear, central nervous system, blood, skin, eye or bone marrow. Target cells comprise or can be generated from induced pluripotent stem cells.

Vectors

Provided herein are vectors for the delivery of a transgene into a target cell. A “vector” is a composition of matter which comprises one or more nucleic acid molecules and which can be used to deliver the one or more nucleic acid molecules to the interior of a target cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, lentivirus, retrovirus, adenovirus, parvovirus (e.g., adeno-associated viruses, AAV), coronavirus, negative strand RNA viruses such as orthomyxovirus (e.g., influenza virus), rhabdovirus (e. g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses may include: avian leukosis-sarcoma, mammalian C-type, B-type viruses, D type viruses, HTLV-BLV group, lentivirus, and spumavirus.

The expression of transgenes is typically achieved by operably linking a nucleic acid (e.g., a transgene) encoding the protein of interest or portions thereof to one or more promoters, and incorporating the construct into a vector. The vector can be suitable for replication and integration in eukaryotes. Typical cloning vectors contain transcription and translation terminators, initiation sequences, and promoters useful for regulation of the expression of the desired nucleic acid sequence. In some embodiments, delivery of a transgene using a vector leads to an increase in the polypeptide encoded by the transgene at the transcript or protein level.

Lentiviral Vectors

In some embodiments, the vector for the delivery of a transgene into a target cell is a lentiviral vector. Suitable lentiviral vectors for use in the compositions and methods disclosed herein include, but are not limited to human immunodeficiency virus (HIV-1, HIV-2), feline immunodeficiency virus (FIV), simian immunodeficiency virus (SIV), bovine immunodeficiency virus (BIV), and equine infectious anemia virus (EIAV). In one embodiment, the vector is made safer by separating the necessary lentiviral genes (e.g., gag and pol) onto separate vectors. In another embodiment, the vector is made safer by replacing certain lentiviral sequences with non-lentiviral sequences. Thus, lentiviral vectors of the present invention may contain partial (e.g., split) gene lentiviral sequences and/or non-lentiviral sequences (e.g., sequences from other retroviruses) as long as their functions (e.g., viral titer, infectivity, integration and ability to confer sufficient levels and duration of therapeutic gene expression) are not substantially reduced.

In order to increase their target cell range and to facilitate concentration by centrifugation, the lentiviral vectors of the invention can be pseudotyped with an envelope protein, such as the vesicular stomatitis virus G-protein (VSV-G), using known techniques in the art (see e.g., Chesebro et al. (1990) J. Virol. 64 (1): 215-221; Naldini et al. (1996), supra; U.S. Pat. No. 5,665,577 (Sodroski et al.); and WO 97/17457 (Salk Institute). The lentiviral gene delivery system also can be used in conjunction with a suitable packaging system able to produce high titers of replication-incompetent lentiviral-based retroviruses.

Adenoviral Vectors

In some embodiments, the vector for the delivery of a transgene into a target cell is a adenoviral vector. The term “adenovirus vectors” refer to constructs containing adenovirus sequences sufficient to (a) support packaging of the construct and (b) express a polynucleotide that has been cloned therein in a sense or antisense orientation. A recombinant adenovirus vector comprises a genetically engineered form of an adenovirus.

In some embodiments, the adenoviral vector has the early genes E1 and/or E3 deleted. E1 may then be supplied by adenovirus packaging lines; its deletion from the viral vector renders the virus replication incompetent. E3 is involved in evading host immunity and is not essential for virus production. Deletion of these two components results in a transgene packaging capacity of >8 Kb. In some embodiments, the E2b gene is also deleted. In some embodiments, the transgene inserted into the E1 region.

The adenoviral vector may be based on any adenovirus serotype or subgroup. Over 50 different adenoviral serotypes are known, grouped into six species. In some embodiments, the viral vector is based on human adenovirus type 2 or human adenovirus type 5. In one embodiment, the adenoviral vector is based on adenovirus serotype 5 (Ad5) Ad5-based vectors use the Coxsackie-Adenovirus Receptor (CAR) and the Alpha and Beta integrins to enter cells. Ad5-based vectors comprise, inter alia, Ad5 ITRs (which are distinct from the transposon ITRs) and the Ad5 virus packaging signal. Adenovirus Ad5 vectors are well known in the art. They have been modified to allow infection into new cell types where CAR is absent. The virus may also be modified genetically by exchanging the fibre protein with that from another adenovirus serotype (such as AD35) to infect via certain surface receptors. An adenovirus may also be targeted to new cell types by the addition of new amino acid sequences with new binding specificities into the hypervariable region of the adenovirus Hexon protein. An example of this is the additional of a region of the VSVG glycoprotein coding sequence into the hexon hypervariable loops to provide broad virus tropism.

Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Adeno-Associated Viral Vectors

In one embodiment, the viral vector is a recombinant AAV (rAAV). AAV particles comprise a linear, single-stranded AAV nucleic acid genome associated with an AAV capsid protein coat. AAV is incapable of replication without a helper virus which can be an adenovirus, vaccinia or herpes virus. In absence of a helper virus, AAV inserts its genome in the host cell chromosome assuming a latent state. Subsequent infection by the helper virus rescues the latent integrated copy which then replicates to produce infectious viral progeny.

Recombinant AAV (rAAV) vectors comprise a recombinant viral genome and capsid proteins. The rAAV genome comprising the transgene(s) can be assembled from polynucleotides encoding the transgene(s), suitable regulatory elements, and viral elements necessary for packaging the rAAV genome. General methods for construction of rAAV genomes are known in the art. The AAV based expression vector may be composed of the AAV inverted terminal repeats (ITRs) flanking a restriction site that can be used for subcloning of the transgene, either directly using the restriction site available, or by excision of the transgene with restriction enzymes followed by polishing the ends and ligation into the AAV expression vector, optionally using linkers. A transgene may be integrated in the AAV based expression vector along with one or more expression control elements including, for example an enhancer, promoter, and/or a post transcriptional regulatory sequence (PRE), flanked by AAV ITRs.

Methods for making rAAV vectors having a specific capsid protein are known in the art. Viral particles can be made by providing the components required for packaging the rAAV genome in a capsid in trans, or required components may be provided by an engineered host cell. Both of the methods use standard molecular biology techniques known to persons skilled in the art. Some or all of the required elements can be either under the control of an inducible or a constitutive promoter. The recombinant AAV genome, rep sequences, cap sequences, and helper functions for producing the rAAV may be delivered to the packaging host cell using any appropriate genetic element (vector). Typically, the recombinant AAVs are produced by transfecting a host cell with a recombinant AAV genome (comprising a transgene) to be packaged into AAV particles, an AAV helper function vector, and an accessory function vector. An AAV helper function vector encodes the AAV helper function sequences (i.e., rep and cap), which function in trans for productive AAV replication and encapsidation. The accessory function vector typically encodes the nucleotide sequences for non-AAV derived viral and/or cellular functions that are required for AAV replication including, without limitation, those elements involved in activation of AAV gene transcription, stage specific AAV mRNA splicing, AAV DNA replication, synthesis of cap expression products, and AAV capsid assembly.

As used herein, the terms “AAV1,” “AAV2,” “AAV3,” “AAV4,” and the like refer to AAV vectors containing inverted terminal repeats (ITR) from AAV1, AAV2, AAV3, or AAV4, respectively, as well as capsid proteins from AAV1, AAV2, AAV3, or AAV4, respectively. The terms “AAV2/1,” “AAV2/8,” “AAV2/9,” and the like refer to pseudotyped AAV vectors containing ITRs from AAV2 and capsid proteins from AAV1, AAV8, or AAV9, respectively.

The AAV vectors described herein generally comprise a rAAV genome encoding one or more transgenes operably linked to one or more regulatory elements in a manner that permits transgene transcription, translation, and/or expression in a target cell or a target tissue and is flanked by 5′ and 3′ ITRs. ITR sequences are typically about 145 bp in length. The AAV ITR sequences can be modified, e.g., by the insertion, deletion or substitution of one or more nucleotides by using standard molecular biology techniques provided the modification of the ITR sequence does not interfere with AAV vector function (such as efficient encapsidation of the rAAV genome). The AAV ITRs may be derived from any of the several AAV serotypes. The AAV ITR sequences at 3′ and 5′ can be identical or derived from different AAV serotype.

The expression control elements or regulatory elements operably linked to the transgene may include a promoter or enhancer, such as the chicken beta actin promoter or cytomegalovirus enhancer, among others described herein. The recombinant AAV genome is generally encapsidated by capsid proteins (e.g., from the same AAV serotype as that from which the ITRs are derived or from a different AAV serotype from that which the ITRs are derived. Components of exemplary AAV vectors that may be used in conjunction with the compositions and methods of the disclosure are described herein.

Any AAV serotype or combination of AAV serotypes can be used in the methods and compositions of the present disclosure. Because the methods and compositions of the present disclosure are for the treatment and cure of neurodegenerative diseases or disorders, AAV serotypes that target at least the central nervous system can be used in some embodiments and include but are not limited to AAV1, AAV2, AAV4, AAV5, AAV6, AAV7, AAV8, AAV9, and AAV10.

Regulatory Sequences

The viral vector may comprise expression control elements that are operably linked to the transgene(s) in a manner which permits its transcription, translation and/or expression in a cell infected with the virus. As used herein, “operably linked” refers to a relationship between two or more nucleic acid sequences where certain nucleic acid sequences (e.g., control elements) influence characteristics of another nucleotide sequence (e.g., influencing expression of a transgene). Operably linked sequences include both expression control elements that are included in or are contiguous with the G transgene, and expression control elements that act in trans or at a distance to control expression of the transgene. Expression control elements as used herein include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. For example, as used herein, a nucleic acid sequence and regulatory sequences are considered to be operably linked when they are covalently linked in such a way as to place the expression or transcription of the nucleic acid sequence under the influence or control of the regulatory sequences.

The promoter operably liked to a transgene can either be inducible or constitutive. Inducible promoters allow regulation of gene expression and can be regulated by exogenously circumstances or compounds. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system; a ecdysone insect, a tetracycline-repressible system. Constitutive promoters are unregulated promoters that allow for continual transcription of its associated gene. Examples of constitutive promoters include, without limitation, a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter and a β-actin promoter,

In embodiments, a native promoter or fragment thereof for the transgene may be used if the expression of the transgene to mimic the native expression is preferred. In another embodiment, a tissue specific promoter is used to allow expression in specific tissues for targeted gene therapy. Tissue specific promoters such allow the protein to express in the specific tissue desired.

Reporter Sequences

In some embodiments, the vector for the delivery of one or more transgenes into a target cell can also contain either a selectable marker gene, a reporter gene, or both to facilitate identification and selection of expressing cells from the population of cells sought to be transfected or transduced through vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neomycin and the like. Reporter proteins are proteins that can be assayed by detecting characteristics of the reporter protein, such as enzymatic activity or spectrophotometric characteristics, or indirectly, such as with antibody-based assays. Examples of reporter gene products that are readily detectable include, but are not limited to, puromycin resistance marker (pac), 3-galactosidase, luciferase, orotidine 5′-phosphate decarboxylase (URA3), arginine permease CAN1, galactokinase (GAL1), beta-galactosidase (LacZ), or chloramphenicol acetyl transferase (CAT).

Non-Viral Delivery Methods

In some embodiments, non-viral delivery methods are used for the delivery of a transgene to a target cell. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Transgenes

Provided herein are compositions and methods for stimulating the formation of one or more auditory hair cells from one or more targets cells by administering a vector comprising one or more transgenes to the one or more targets cells.

In one aspect, the transgene encodes a protein selected from the group consisting of Six1, Eya1, Atoh1, Pou4f3, Gfi1, Pbx1, Fgf8, Dusp6, Pbx1, Vangl1/Vangl2/Celsr1, Ptk7, and Gata3. In some embodiments, the transgene encodes a protein selected from the group consisting of Six1, Eya1, Atoh1, Pou4f3, and Gfi1. In some embodiments, Six1, Eya1, Atoh1, Pou4f3, and/or Gfi1 are human proteins. In some embodiments, Six1, Eya1, Atoh1, Pou4f3, and/or Gfi1 are murine proteins. In some embodiments, Six1, Eya1, Atoh1, Pou4f3, and/or Gfi1 are rat proteins. The sequence of human Six1 is provided by SEQ ID NO: 1. The sequence of human Eya1 is provided by SEQ ID NO:2. The sequence of human Atoh1 is provided by SEQ ID NO 3 The sequence of human Pou4f3 is provided by SEQ ID NO:4 The sequence of human Gfi1 is provided by SEQ ID NO:5. See Table 1.

In some embodiments, the transgene encodes a protein that is at least 80%, at least 85%, at least 90%, at least 95%, at least 96%, at least 97%, at least 98%, or at least 99% identical to any of the sequences of SEQ ID NOs:1-5.

As used herein, the term “identity” refers to sequence identity between two nucleic acid molecules or polypeptides. Identity can be determined by comparing a position in each sequence, which may be aligned for purposes of comparison. A degree identity between nucleic acid or amino acid sequences is a function of the number of identical or matching nucleotides or amino acids at shared positions. For example, polypeptides having at least 85%, at least 90%, at least 95%, at least 96%, at least 97%, at least 98%, or at least 99% identity to specific polypeptides described herein and preferably exhibiting substantially the same functions, as well as polynucleotides encoding such polypeptides, are contemplated. Methods and computer programs for determining both sequence identity and similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

Also provided are amino acid substitution variants of the proteins disclosed herein. These variants have at least one amino acid residue replaced by a different residue that has similar side chain properties. Amino acids can be grouped according to similarities in the properties of their side chains (see Lehninger, BIOCHEMISTRY (2nd ed., Worth Publishers, New York, 1975):

(1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q);
(3) acidic: Asp (D), Glu (E);
(4) basic: Lys (K), Arg (R), His (H).

As such, a non-limiting example for a conservative amino acid substitution is one that replaces a non-polar amino acid with another non-polar amino acid.

Alternatively, naturally occurring residues can be divided into groups based on common side-chain properties:

(1) hydrophobic: Ala (A), Val (V), Leu (L), Ile (I), Met (M);
(2) neutral hydrophilic: Ser (S), Thr (T), Cys (C), Asn (N), Gln (Q);
(3) acidic: Asp (D), Glu (E);
(4) basic: Lys (K), Arg (R), His (H);
(5) residues that influence chain orientation: Gly (G), Pro (P);
(6) aromatic: Phe (F), Trp (W), Tyr (Y).

As such, a non-limiting example for a conservative amino acid substitution is one that replaces a hydrophobic amino acid with another hydrophobic amino acid.

TABLE 1
Amino acid sequences

| SEQ | | |
|---|---|---|
| ID | | |
| NO | Protein | Amino acid sequence |
| 1 | Six1 | MSMLPSFGFTQEQVACVCEV |
| | | LQQGGNLERLGRFLWSLPAC |
| | | DHLHKNESVLKAKAVVAFHR |
| | | GNFRELYKILESHQFSPHNH |
| | | PKLQQLWLKAHYVEAEKLRG |
| | | RPLGAVGKYRVRRKFPLPRT |
| | | IWDGEETSYCFKEKSRGVLR |
| | | EWYAHNPYPSPREKRELAEA |
| | | TGLTTTQVSNWFKNRRQRDR |
| | | AAEAKERENTENNNSSSNKQ |
| | | NQLSPLEGGKPLMSSSEEEF |
| | | SPPQSPDQNSVLLLQSNMGH |
| | | ARSSNYSLPGLTASQPSHGL |
| | | QAHQHQLQDSLLGPLTSSLV |
| | | DLG |
| 2 | Eya1 | MEMQDLTSPHSRLSGSSESP |
| | | SGPKLDSSHINSTSMTPNGT |
| | | EVKTEPMSSSEIASTAADGS |
| | | LDSFSGSALGSSSFSPRPAH |
| | | PFSPPQIYPSKSYPHILPTP |
| | | SSQTMAAYGQTQFTTGMQQA |
| | | TAYATYPQPGQPYGISSYGA |
| | | LWAGIKTESGLSQSQSPGQT |
| | | GFLSYGTSFGTPQPGQAPYS |
| | | YQMQGSSFTTSSGLYSGNNS |
| | | LTNSSGFNSSQQDYPSYPGF |
| | | GQGQYAQYYNSSPYPAHYMT |
| | | SSNTSPTTPSTNATYQLQEP |
| | | PSGVTSQAVTDPTAEYSTIH |
| | | SPSTPIKETDSERLRRGSDG |
| | | KSRGRGRRNNNPSPPPDSDL |
| | | ERVFIWDLDETIIVFHSLLT |
| | | GSYANRYGRDPPTSVSLGLR |
| | | MEEMIFNLADTHLFFNDLEE |
| | | CDQVHIDDVSSDDNGQDLST |
| | | YNFGTDGFPAAATSANLCLA |
| | | TGVRGGVDWMRKLAFRYRRV |
| | | KEIYNTYKNNVGGLLGPAKR |
| | | EAWLQLRAEIEALTDSWLTL |
| | | ALKALSLIHSRTNCVNILVT |
| | | TTQLIPALAKVLLYGLGIVF |
| | | PIENIYSATKIGKESCFERI |
| | | IQRFGRKVVYVVIGDGVEEE |
| | | QGAKKHAMPFWRVSSHSDLM |
| | | ALHHALELEYL |
| 3 | Atoh1 | MSRLLHAEEWAEVKELGDHH |
| | | RHPQPHHVPPLTPQPPATLQ |
| | | ARDLPVYPAELSLLDSTDPR |
| | | AWLTPTLQGLCTARAAQYLL |
| | | HSPELGASEAAAPRDEADSQ |
| | | GELVRRSGCGGLSKSPGPVK |
| | | VREQLCKLKGGVVVDELGCS |
| | | RQRAPSSKQVNGVQKQRRLA |
| | | ANARERRRMHGLNHAFDQLR |
| | | NVIPSFNNDKKLSKYETLQM |
| | | AQIYINALSELLQTPNVGEQ |
| | | PPPPTASCKNDHHHLRTASS |
| | | YEGGAGASAVAGAQPAPGGG |
| | | PRPTPPGPCRTRFSGPASSG |
| | | GYSVQLDALHFPAFEDRALT |
| | | AMMAQKDLSPSLPGGILQPV |
| | | QEDNSKTSPRSHRSDGEFSP |
| | | HSHYSDSDEAS |
| 4 | Pou4f3 | MMAMNAKQPFGMHPVLQEPK |
| | | FSSLHSGSEAMRRVCLPAPQ |
| | | LQGNIFGSFDESLLARAEAL |
| | | AAVDIVSHGKNHPFKPDATY |
| | | HTMSSVPCTSTSPTVPISHP |
| | | AALTSHPHHAVHQGLEGDLL |
| | | EHISPTLSVSGLGAPEHSVM |
| | | PAQIHPHHLGAMGHLHQAMG |
| | | MSHPHAVAPHSAMPACLSDV |
| | | ESDPRELEAFAERFKQRRIK |
| | | LGVTQADVGAALANLKIPGV |
| | | GSLSQSTICRFESLTLSHNN |
| | | MIALKPVLQAWLEEAEAAYR |
| | | EKNSKPELFNGSERKRKRTS |
| | | IAAPEKRSLEAYFAIQPRPS |
| | | SEKIAAIAEKLDLKKNVVRV |
| | | WFCNQRQKQKRMKYSAVH |
| 5 | Gfi1 | MPRSFLVKSKKAHSYHQPRS |
| | | PGPDYSLRLETVPAPGRAEG |
| | | GAVSAGESKMEPRERLSPDS |
| | | QLTEAPDRASASPNSCEGSV |
| | | CDPCSEFEDFWRPPSPSVSP |
| | | ASEKSLCRSLDEAQPYTLPF |
| | | KPYAWSGLAGSDLRHLVQSY |
| | | RQCSALERSAGLSLFCERGS |
| | | EPGRPAARYGPEQAAGGAGA |
| | | GQPGRCGVAGGATSAAGLGL |
| | | YGDFAPAAAGLYERPSTAAG |
| | | RLYQDHGHELHADKSVGVKV |
| | | ESELLCTRLLLGGGSYKCIK |
| | | CSKVFSTPHGLEVHVRRSHS |
| | | GTRPFACEMCGKTFGHAVSL |
| | | EQHKAVHSQERSFDCKICGK |
| | | SFKRSSTLSTHLLIHSDTRP |
| | | YPCQYCGKRFHQKSDMKKHT |
| | | FIHTGEKPHKCQVCGKAFSQ |
| | | SSNLITHSRKHTGFKPFGCD |
| | | LCGKGFQRKVDLRRHRETQH |
| | | GLK |

In some embodiments, more than one transgene is delivered to a target cell. In some embodiments, a group of transgenes is delivered to a target cells, wherein the transgenes are selected from the group consisting of Six1, Eya1, Atoh1, Pou4f3, and Gfi1.

In some embodiments, a vector comprises one or more nucleic acids encoding for more than one transgene.

Methods of Treatment

Provided herein are compositions and methods for treating and preventing hearing loss, for treating and preventing a disorder associated with loss, damage, or absence of sensory auditory hair cells, and/or for improving auditory function in a subject in need thereof. Also provided herein are compositions and methods for the generation of auditory hair cells that allow perception of stimuli in a subject in need thereof.

The terms “treat,” “treated,” “treating,” or “treatment” as used herein refer to a therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms, diminishment of the extent of the condition or disorder, stabilization (i.e., not worsening) of the state of the condition or disorder, slowing of the progression of the condition or disorder, amelioration of the state of the condition or disorder, or improvement of the condition or disorder. Treatment includes eliciting a clinically significant response without excessive levels of side effects. The reduction in severity or recurrence of hearing loss or a disorder associated with loss, damage, or absence of auditory hair cells can be about a 10, 20, 30, 40, 50, 60, 70, 80, 90, 100%, or any amount of reduction in between. A “therapeutically effective amount” means an amount that, when administered to a subject, is effective in producing the desired therapeutic effect.

The patient may be asymptomatic and/or may have a predisposition to the disease. As such, in one embodiment the disclosure provides methods of reducing the reducing the likelihood, delaying, or preventing the onset of hearing loss and/or a disorder associated with loss, damage, or absence of auditory hair cells. A “prophylactically effective amount” is an amount that prevents, reduces, and/or delays the onset of one or more symptoms of the condition or disorder. Prophylactic and preventive are used interchangeably herein. The delay in onset, incidence, severity, or recurrence of hearing loss or a disorder associated with loss, damage, or absence of auditory hair cells, can be about a 10, 20, 30, 40, 50, 60, 70, 80, 90, 100%, or any amount of reduction in between.

As used herein the term “hearing loss” is intended to mean any reduction in a subject's ability to detect sound. Hearing loss is defined as a 10 decibel (dB) standard threshold shift or greater in hearing sensitivity for two of 6 frequencies ranging from 0.5-6.0 (0.5, 1, 2, 3, 4, and 6) kHz (cited in Dobie, R. A. (2005) Audiometric Threshold Shift Definitions: Simulations and Suggestions, Ear and Hearing 26(1) 62-77). Hearing loss can also be only high frequency, and in this case would be defined as 5 dB hearing loss at two adjacent high frequencies (2-6 kHz), or 10 dB at any frequency above 2 kHz. Non-limiting examples of types of hearing loss include. 1) ototoxicity caused by chemical or pharmaceutical agents, for example, antineoplastic agents such as cisplatin or related compounds, aminoglycosides, antineoplastic agents, and other chemical ototoxic agents; 2) noise induced hearing loss, either from acoustic trauma or blast injury; 3) therapeutic radiation, 4) viral infections of the inner ear, such as Herpes Simplex or other viruses or infectious agents (such as Lyme Disease) that can cause inner ear hearing loss; 5) autoimmune inner ear diseases: 6) genetic hearing losses that may have an apoptotic component; 7) inner ear barotrauma such as diving or acute pressure changes; 8) physical trauma such as that caused by head injury, or surgical trauma from surgical intervention in the inner ear, 9) inflammation or other response to administration of other inner ear regenerative compounds or gene therapy techniques; and 10) age-related hearing loss, which is the gradual onset of hearing loss with increasing age. Symptoms of hearing loss include, but are not limited to trouble understanding speech, listening to television or radio at high volume, tinnitus, and asking people to repeat themselves.

As used herein, the phrase “improving auditory function” means improving, by at least 10%, the sensitivity to sound of an inner ear by treating the inner ear in accordance with the methods of the present disclosure, or effecting any measurable improvement in the sensitivity to sound of an inner ear that is completely unresponsive to sound prior to treatment in accordance with the present invention. The sensitivity to sound of the treated inner ear can be measured by any art-recognized means (such as the auditory brainstem response) and compared to the sensitivity to sound of a control inner ear that is not treated in accordance with the present invention and which is cultured under substantially the same conditions as the treated inner ear.

A “subject” or “patient” can be an adult subject or a pediatric subject. Pediatric subjects include subjects ranging in age from birth to eighteen years of age Thus, pediatric subjects of less than about 10 years of age, five years of age, two years of age, one year of age, six months of age, three months of age, one month of age, one week of age or one day of age are also included as subjects. The subject may be male or female. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.) Thus, veterinary uses and medical formulations are contemplated herein.

In general, there are two approaches to gene therapy in humans. For in vivo gene therapy, a vector encoding the gene of interest can be administered directly to the patient. Alternatively, in ex vivo gene therapy, cells are removed from a patient and treated with a vector to express the gene of interest. In the ex vivo method of gene therapy, the treated cells are then re-administered to the same or to a different patient. Partially and/or fully differentiated auditory hair cells, e.g., generated by the methods described herein, can be transplanted or implanted, such as in the form of a cell suspension, into the ear by injection, such as into the luminae of the cochlea. Injection can be, for example, through the round window of the ear or through the bony capsule surrounding the cochlea. The cells can be injected through the round window into the auditory nerve trunk in the internal auditory meatus or into the scala tympani.

Provided herein is a method for treating and preventing hearing loss in a subject in need thereof, the method comprising administering to a target cell in the organ of Corti in a subject one or more vectors comprising one or more nucleic acids encoding one or more gene products. The one or more vectors may be administered simultaneously or sequentially.

Provided herein is a method for treating and preventing hearing loss in a subject in need thereof, the method comprising administering to a target cell in the organ of Corti in a subject one or more vectors comprising one or more nucleic acids encoding one or more gene products, wherein the gene products are selected from the group consisting of Six1, Eya1, Atoh1, Pou4f3, and Gfi1.

Provided herein is a method for treating and preventing a disorder associated with loss, damage, or absence of auditory hair cells, the method comprising administering to a target cell in the organ of Corti in a subject one or more vectors comprising one or more nucleic acids encoding one or more gene products, wherein the gene products are selected from the group consisting of Six1, Eya1, Atoh1, Pou4f, and Gfi1.

Provided herein is a method for regenerating auditory hair cells in a subject in need thereof, the method comprising administering to a target cell in the organ of Corti in a subject one or more vectors comprising one or more nucleic acids encoding one or more gene products, wherein the gene products are selected from the group consisting of Six1, Eya1, Atoh1, Pou4f3, and Gfi1.

In some embodiments, the vector administered to the target cell in the organ of Corti in the subject comprises a nucleic acid encoding Six1. In some embodiments, the one or more vectors administered to the target cell in the organ of Corti in the subject comprise one or more nucleic acids encoding Six1 and Eya1. In some embodiments, the one or more vectors administered to the target cell in the organ of Corti in the subject comprise one or more nucleic acids encoding Six1, Atoh1, and Pou4f3. In some embodiments, the one or more vectors administered to the target cell in the organ of Corti in the subject comprise one or more nucleic acids encoding Six1, Atoh1, Pou4f3, and Gfi1.

Provided herein is a method for treating and preventing hearing loss in a subject in need thereof, the method comprising administering to a subject a cell transduced with one or more vectors comprising one or more nucleic acids encoding one or more gene products, wherein the gene products are selected from the group consisting of Six1, Eya1, Atoh1, Pou4f3, and Gfi1.

Provided herein is a method for treating and preventing a disorder associated with loss, damage, or absence of auditory hair cells, the method comprising administering to a subject a cell transduced with one or more vectors comprising one or more nucleic acids encoding one or more gene products, wherein the gene products are selected from the group consisting of Six1, Eya1, Atoh1, Pou4f3, and Gfi1.

Provided herein is a method for regenerating auditory hair cells in a subject in need thereof, the method comprising administering to a subject a cell transduced with one or more vectors comprising one or more nucleic acids encoding one or more gene products, wherein the gene products are selected from the group consisting of Six1, Eya1, Atoh1, Pou4f3, and Gfi11.

In some embodiments, the cell administered the subject is transduced with a vector comprising a nucleic acid encoding Six1. In some embodiments, the cell administered the subject is transduced with one or more vectors comprising one or more nucleic acids encoding Six1 and Eya1. In some embodiments, the cell administered the subject is transduced with one or more vectors comprising one or more nucleic acids encoding Six1, Atoh1, and Pou4f3. In some embodiments, the cell administered the subject is transduced with one or more vectors comprising one or more nucleic acids encoding Six1, Atoh1, Pou4f3, and Gfi1.

In some embodiments, the target is an inner ear supporting cell. In some embodiments, the supporting cell is an inner border, Deiters' cell, inner pillar cell, outer pillar cell, inner phalangeal cell, or Hensen's cell.

Provided is a viral vector for use in the treatment of hearing loss in a subject in need thereof, the viral vector comprising one or more polynucleotides encoding one or more gene products, wherein the gene products are selected from the group consisting of Six1, Eya1, Atoh1, Pou4f3, and Gfi1.

Provided is a cell for use in the treatment of hearing loss in a subject in need thereof, wherein the cell is transduced with one or more vectors comprising one or more polynucleotides encoding one or more gene products, wherein the gene products are selected from the group consisting of Six1, Eya1, Atoh1, Pou4f3, and Gfi1.

In some embodiments, the methods include steps of selecting a subject at risk of auditory hair cell loss and/or a subject with auditory hair cell loss. Alternatively or in addition, the methods include steps of selecting a subject at risk of sensorineural hearing loss and/or a subject with sensorineural hearing loss. A subject experiencing or at risk for developing hearing loss is a candidate for the treatment methods described herein. A human subject having or at risk for developing a hearing loss can hear less well than the average human being, or less well than a human before experiencing the hearing loss. For example, hearing can be diminished by at least 5, 10, 30, 50% or more.

Provided herein is a method of treating a subject who has hearing loss as a result of loss, damage, or absence of auditory hair cells, the method comprising:

(a) identifying a subject who has hearing loss as a result of loss, damage, or absence of auditory hair cells;
(b) administering to a target cell in the organ of Corti in the subject one or more vectors comprising one or more nucleic acids encoding (i) Six1, (ii) Six1 and Eya1; (iii) Six1, Atoh1, and Pou4f3; or (iv) Six1, Atoh1, Pou4f3, and Gfi1; thereby treating the hearing loss as a result of loss, damage, or absence of auditory hair cells in the subject.

Provided herein is a method of treating a subject who has a disorder associated with loss, damage, or absence of auditory hair cells, the method comprising:

(a) identifying a subject who has a disorder associated with loss, damage, or absence of auditory hair cells;
(b) administering to a target cell in the organ of Corti in the subject one or more vectors comprising one or more nucleic acids encoding (i) Six1, (ii) Six1 and Eya1; (iii) Six1, Atoh1, and Pou4f3; or (iv) Six1, Atoh1, Pou4f3, and Gfi1; thereby treating the a disorder associated with loss, damage, or absence of auditory hair cells in the subject.

Pharmaceutical Compositions

Provided herein is a pharmaceutical composition comprising any of the vectors described herein and a pharmaceutically acceptable carrier, diluent, excipient, or buffer. In some embodiments, the pharmaceutically acceptable carrier, diluent, excipient, or buffer is suitable for use in a subject, for example, a human. The pharmaceutical compositions can be delivered to a subject, so as to allow production of a gene product in an inner ear cell of the subject. Pharmaceutical compositions comprise sufficient genetic material that allows the recipient to produce an effective amount of a gene product that reduces or prevents auditory hair cell damage. In some embodiments, the pharmaceutical compositions comprise sufficient genetic material that allows the recipient to produce an effective amount of a gene product that treats or prevents hearing loss, treats or prevents a disorder associated with loss, damage, or absence of auditory hair cells, and/or regenerates auditory hair cells in a subject.

The compositions may be administered alone or in combination with at least one other agent, such as stabilizing compound, which may be administered in any sterile, biocompatible pharmaceutical carrier, including, but not limited to, saline, buffered saline, dextrose, and water. In some embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. The preparation of pharmaceutically acceptable carriers, excipients and formulations containing these materials is described in, e.g., Remington: The Science and Practice of Pharmacy, 22nd edition, Loyd V. Allen et al, editors, Pharmaceutical Press (2012).

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Methods of Administration

In some aspects, the compositions disclosed herein can be administered locally to the inner ear. For example, using injection into the luminae of the cochlea, into the auditory nerve trunk in the internal auditory meatus, and/or into the scala tympani. Such methods can also include, for example, administered to the middle, or the inner ear, or both, e.g., using a catheter or pump.

In some embodiments, is administered intravenously, intrathecally, intratypmanically, via round window administration, via semicircular canal delivery, via injection through the cochlear capsule, or via stapedotomy.

The effective amounts and schedules for administering the vectors or the pharmaceutical compositions described herein can be determined empirically and making such determinations is within the skill in the art. The dosage ranges for administration are those large enough to produce the desired effect in which one or more symptoms of the disease or disorder are affected (e.g., reduced or delayed). In embodiments, the dosage is not so large as to cause substantial adverse side effects, such as unwanted cross-reactions, unwanted cell death, and the like. Generally, the dosage will vary with the type of vector and transgene, the species, age, body weight, general health, sex and diet of the subject, the mode and time of administration and severity of the particular condition and can be determined by one of skill in the art. The dosage can be adjusted by the individual physician in the event of any contraindications Dosages can vary and can be administered in one or more doses.

In some embodiments, a viral vector is administered at about 10⁴, 10⁵, 10⁶, 10⁷, 10⁸, 10⁹, 10¹⁰, 10¹¹ 10¹², 10¹³, 10¹⁴ or 10¹⁵ genome copies per subject. In some embodiments, a viral vector is administered at about 10⁴, 10⁵, 10⁶, 10⁷, 10⁸, 10⁹, 10¹⁰, 10¹¹ 10¹², 10¹³, 10¹⁴ or 10¹⁵ genomes/ml.

In some embodiments, the compositions and methods disclosed herein are combined with a second therapy or a device aimed at reducing or preventing hearing loss, at reducing or preventing a disorder associated with loss, damage, or absence of auditory hair cells, and/or at regenerating auditory hair cells in a subject. The compositions and methods disclosed herein maybe be used simultaneously or consecutively with the second therapy or device. Suitable devices include, but are not limited to surgical procedures, hearing aids, and cochlear implants.

It is to be understood that this disclosure is not limited to the particular molecules, compositions, methodologies, or protocols described, as these may vary. Any methods and materials similar or equivalent to those described herein can be used in the practice or testing of embodiments of the disclosure. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entireties. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the disclosure, the following examples of specific embodiments are given. The following examples should not be read to limit or define the entire scope of the invention.

### EXAMPLES

### Example 1: Six1 Cooperates with Distinct Cofactors in Feedforward Loops to Control Lineage-Specific Gene Expression Programs During Progressive Differentiation of the Auditory Sensory Epithelium

Dynamic Changes in Genomic Occupancy by Six1 During Auditory Sensory Cell Fate Commitment

ChIP-seq experiments were performed to investigate the global occupancy of Six1-binding from undifferentiated prosensory progenitors in the auditory sensory epithelium at E13.5 to differentiation at E16.5. To better characterize the chromatin structure associated with Six1, E16.5 was compared with E13.5 cochlea ChIP-seq of the histone mark H3K27ac for enhancers and the H3K27me3 mark for chromatin condensation and transcriptional repression. A total of 14,967 Six1-bound regions and observed clusters with varying levels of enrichment were identified. 5270 regions showed loss of or reduced Six1-occupancy at E16.5 with very weak or no H3K27ac-deposition, while 6616 regions showed new or increased binding at E16.5 with weaker H3K27ac-deposition at E13.5. 2981 Six1-bound sites retained occupancy upon differentiation and had strong H3K27ac-deposition, indicating that these regions are enhancers from E13.5. ˜66% of E16.5 and 37% of E13.5 peaks were marked by H3K27ac, indicting an increase in the proportion of Six1-bound enhancers as differentiation proceeds. These Six1-bound sites were associated with a total of 7558 genes. Among them, 3214 were common to both stages and 1551 or 2793 were E13.5- or E16.5-specific genes respectively. The three clusters of peaks share common genes due to multiple distinct peaks per gene.

Further, stage-specific differences in the genomic distribution of Six1 peaks were observed. The majority (˜96%) of E13.5 peaks were intronic and intergenic and 84% of the precursor-transient peaks were distal regions >5 kb from TSSs (transcriptional start sites) of the nearest genes. By E16.5, the number of intronic peaks remained similar, but the proportion of intergenic Six1 sites was drastically reduced, while the proportion of promoter-TSSs sites was increased.

Thus, this analysis revealed the highly dynamic nature of Six1-binding patterns during cell fate induction and subsequent differentiation in the auditory sensory epithelium development. The higher density of E16.5 peaks in the vicinity of the TSSs reflects the functional relevance of these sites in regulating differentiation genes.

Six1 Binds to a Broad Set of Key Loci to Drive Sensory Epithelium Formation

GREAT and Gene Ontology analyses revealed overrepresentation of genes related to inner-ear/cochlea development in Six1 targets. Notably, differentiation peaks were significantly enriched for terms related to molecular function of voltage-gated chloride channel activity, actin filament binding, and single-stranded RNA binding.

The global analyses indicated that Six1-occupancy to putative CREs is dynamic over the time. To illustrate this behavior, Six1's associations were highlighted with several targets that are active in E13.5 cochlea (without H3K27me3-deposition) and involved in the Wnt, Notch, Shh, and Fgf signaling pathways that are crucial for prosensory primordium specification and both auditory hair cell and supporting cell fate selection. The Wnt signaling mediator Lgr5 is expressed in prosensory progenitors and maintained in a subset of supporting cells during differentiation. Lgr5^{d-} cells are capable of differentiating into hair cells in response to Wnt signaling. Three Six1-bound regions with H3K27ac-deposition at the Lgr5: two persistent (one promoter-proximal and one distal −d-66-kb) were identified and one precursor-transient −d-94-kb, which were confirmed by ChIP-qPCR. Similar dynamics were observed in Six1 peaks at Wnt5a/Tcfs, and in the Notch (Notch1,2/Jag1/Rbpj/Hes1,5/Hey1), Shh (Gli3/Mycn/Tulp3), BMP (Bmp2,3,4,5,6,7,8/Bmper/Bmpr1a,1b), and Fgf (Fgfr1,2,3/FgfJ1,7,8,9,10,16,17,18,20,21 and Dusp1,4,6,7,10,11,14,16,18,26) pathways. Stage-associated changes in Six1 peaks were also observed at loci encoding TFs that are essential for sensory epithelium development and cell fate induction, including Six1 itself, Sox2/4, Pax2, Hes1 and Hey1.

In summary, the Six1 ChIP-seq demonstrates the time course of binding dynamics of this key TF in both hair and supporting cell fate selection and subsequent differentiation, thus uncovering a broad role for Six1 in auditory sensory epithelium development.

Six1 Occupies Enhancer Repertoire to Regulate Sequential Induction of Key TFs that then Engage in Protein Complexes

The hair cell fate is induced upon activation of Atoh1, which regulates the expression of downstream TFs Pou4f3 and Gfi1. Before the onset of hair cell differentiation-E14.5, all three genes had H3K27me3-deposition at E13.5. Atoh1 is a target of Six1 based on the dual criteria of changes in Atoh expression in response to Six1 loss- or gain-of-function experiments and Six1-binding to the 1.4-kb 3′-Atoh1 autoregulatory enhancer. ChIP-seq revealed Six1-binding to this region (peak-2, increased by E16.5). Six1-occupancy to a promoter-differentiation peak (peak-1a,b) was also identified. This region has been previously reported as a target of the Notch mediator Hes/Hey repressor families for supporting cell fate selection. Notably, two distal regions—a precursor-transient peak-3˜_{d-}53.5-kb and a persistent/differentiation peak-4˜_{d-}70-kb—were also occupied by Six1. ChIP-qPCR confirmed stage-related changes in Six1-occupancy and revealed a significant increase in Six1-binding to peak-1a and peak-4 by E16.

To further examine the functional roles of the Six1-bound Atoh1 CREs, the activity of the two novel distal regions was examined using mouse transient transgenic assays. The precursor transient 500-bp of Atoh1+53500 had no activity in E17.5-E18.5 cochlea (3/3 transgenic lines), indicating that this precursor Six1-occupancy may “prime” Atoh1 by limiting binding to other TFs. In contrast, a 500-bp of Atoh1+70000 drove hair-cell-restricted expression in all inner-ear sensory organs in all 5 transgenic lines with 3/5 lines showing a mosaic expression pattern, which often occurs in pronuclear injection where DNA is integrated in a two-cell or later stage embryo. Thus, a novel Six1-bound distal Atoh1 enhancer was identified. It was further found that Six1 targets both proximal and distal CREs to regulate Atoh1 expression “in time and space” to specify hair cell fate.

At the Pou4f3 locus, a distal persistent peak-1˜-15-kb was identified and confirmed by ChIP-qPCR with stronger enrichment at E16.5 than at E13.5. Gfi1 is a target of Pou4f3 and at the Gfi1 locus, a persistent peak ˜+37-kb and a differentiation peak near the promoter-TSS were identified. In transgenic assays, both Pou43-15000 and Gfi1+37000 drove HC-restricted expression in all sensory organs. Multiple peaks were also identified at Gata3, which was reported to synergize with Atoh1/Pou4f3 to convert supporting cells to hair cells in young mice. Co-immunoprecipitation (coIP) revealed complex formation of Six1 with Atoh1, Pou4f3, Gfi1 or Gata3 in cochlea or 293 cells. Together, these data indicate that Six1 acts in a positive feedforward loop in which it regulates Atoh1, which then forms protein complexes to autoregulate Atoh1 and regulate the expression of downstream TFs Pou4f3 or Gfi1 that then cooperatively control targets through direct binding to CREs/enhancers in order to drive the precise timing of hair cell fate specification and stepwise differentiation.

Further, it was discovered that Six1 pre-occupies CREs of hair-cell-subtype-specific genes at the precursor stage, including inner-hair-cell-specific Calb2 (Calretinin) and outer-hair-cell-specific Slc26a5 (Prestin). Six1-occupancy was also observed in supporting-cell-subtype-specific genes, including S100a (inner-hair, inner-phalangeal/Deiters' cells) and Slc1a3 (GLAST, inner-phalangeal/inner-border cells). Thus, Six1 may engage target sites in chromatin to “prime” the enhancers for later activation.

Six1 Binds DNA at Sites Carrying Consensus Sequences for CTCF/BORIS and RFX

As expected from a direct association of Six1-DNA, the most enriched motif (P=10⁻³¹³⁸ or P=10⁻₂₃₁₁) matched to the Six1/2-binding motifs, the majority of which were enriched at the peak center within ±200-bp. A higher proportion of peaks at E16.5 (˜54%) than E13.5 (˜35%) lacked Six1/2-binding sites, indicating an indirect association of Six1 to DNA through interactions with DNA-binding proteins.

Examining the presence of known motifs revealed that CTCF/BORIS, RFX/X-box (HTH), IRF and NF1/CTF are among the top five most enriched motifs. CTCF/BORIS are essential epigenetic components with a primary role in the organization of global chromatin architecture. CTCF has a role in auditory sensory epithelium development but not in HC formation. The NF1/CTF (CAAT box-binding/nuclear factor-1) is a widely expressed TF that controls DNA transcription and replication. RFX proteins Rfx1/3 were recently reported to have a redundant role in differentiating hair cells at postnatal stage. Other highly overrepresented motifs include SOX, bHLH, homeobox, and TCF proteins that are known to interact with the SIX family proteins. Moreover, novel motifs for ETS, Tlx (NR), Forkhead and TEAD proteins were also significantly enriched. Consistent with the coIP analyses, additional motifs for Atoh1, Gfi1, OCT/POU and GATA were enriched to a lesser degree. This analysis provides insight that potential TFs with these combinatory motifs may act as critical components of Six1-bound CREs functioning in vivo.

Next the examination focused on whether Rfx1/3 collaborate in Six1-DNA interactions due to their importance in differentiated hair cells. Western blot and immunohistochemistry confirmed the expression of Rfx1/3 in the sensory epithelium of E13.5-16.5 cochlea. CoIP analysis revealed complex formation between Six1 and Rfx1/3 or CTCF in cochlea or 293 cells. Comparison of Six1 ChIP-seq data with published Rfx1/3 ChIP-seq in mouse Min6 cells showed 2348 or 1113 of Six1 peaks co-occupied by Rfx1 or Rfx3 respectively (above 70% of them are <5 kb to TSS). 12 common peaks were selected and ChIP-qPCR performed using chromatin from E14.5-15.5 cochleae to confirm in vivo occupancy of Rfx1 or Rfx3 for all 12 regions. As Six1 also occupies proximal-promoter of Rfx1 and Rfx3, Six1 may act in a similar positive feedforward loop to form protein complexes with RFX to synergistically coregulate their targets during differentiation.

Dependence of Enhancer Activity on Co-Binding of Six1-Rfx1/3

To investigate whether Six1-RFX coregulate targets through common CREs, Pbx1 was selected due to the presence of multiple Six1-bound regions at this gene and its unknown function in the inner ear. Six1 occupies two distal regions −+39-kb and −+49-kb at E10.5 and Pbx1+49000 with higher sequence conservation contains two SIX-motifs separated by an RFX-motif (FIG. 4B). ChIP-qPCR confirmed Rfx1/3-binding to this region in both cochlea and 293 cells cotransfected with a reporter transgene driven by a 510-bp of Pbx1+49000 and Six1 or Rfx3 expression plasmid respectively. A 4-bp mutation of each of the predicted SIX-motifs and a 5-bp mutation of the RFX-motif abolished Six1- or Rfx3-binding.

In transgenic embryos, the 510-bp of Pbx1+49000 was active in the otocyst, cochlear hair cells and flanking nonsensory cells (n=7/7 transgenic lines), recapturing the pattern of Pbx1 mRNA expression detected by in situ hybridization. However, β-Gal activity was also found in supporting cells in the sensory epithelium, which is likely due to lack of cooperative interactions with repressive elements that are present in the locus. The 4-bp mutation of SIX-binding sites did not completely disrupt the activity in the otocyst, but did decrease activity in the auditory hair cells (n=3/3 transgenic lines). However, mutation of both SIX:RFX motifs disrupted enhancer activity in the otocyst and cochlear epithelium, including the flanking nonsensory GER (greater epithelial ridge) and Hensen's cells (n=8/8 transgenic lines) and some β-Gal activity was only observed in an ectopic region above the GER toward the roof of the cochlear duct. Similar observation was obtained from vestibular sensory organs. These results suggest that Six1 and RFX proteins act synergistically to coregulate the expression of Pbx1 via direct binding to the SIX:RFX motifs of Pbx1+49000 enhancer.

Consistent with the decreased transgene activity in the cochlear epithelium driven by the SIXmt enhancer, examination of Pbx1 mRNA expression in Six1^{Cko/Cko} cochlea revealed decreased Pbx1 expression in hair cells, GER and Hensen's cells in Six1-deficient cochlea (tamoxifen given from E12.5 using Eya1^{CreER}) compared to control littermates. This further confirms that Pbx1 expression in the cochlea is partly dependent on Six1 activity.

CoIP analysis found that Pbx1 and Six1 also form protein complexes both in vivo and in vitro, which is consistent with the identification of Pbx1-motif in Six1 peaks. Altogether, these results identify Pbx1 as both a functional target and a novel partner TF of Six1, acting in a similar positive feedforward regulation of sensory epithelium development.

Six1 Regulates the Expression of Fgf8 and Effector Dusp6 of the Fgf Signaling in the Sensory Epithelium Through Directly Binding to Cell-Subtype-Specific Enhancers

Next, the activity of Six1-bound CREs in Fgf signaling was characterized, which plays diverse roles in auditory sensory epithelium formation and growth. Fgf8 had previously been identified as a target of Six1 based on its decreased expression in Six1-deficient inner hair cells. Six1 ChIP-seq identified two distal-persistent peaks −+25-kb and −+67-kb and a proximal-differentiation peak −−4.5-kb at Fgf8. Examination of Fgf8+25000 in vivo showed strong activity restricted to Fgf8-expressing inner hair cells (n=9/9 transgenic lines). Expansion of weak activity in outer hair cells is likely due to lack of cooperative interactions with repressive elements in the locus. This region contained two Six1/2-motifs separated by a GATA and a bHLH-binding E-box motifs. A LacZ or GFP reporter transgene driven by a 714-bp fragment of Fgf8+25000 was generated and introduced two mutations of the predicted SIX-motifs (SIXmt1 and SIXmt2). ChIP-qPCR using chromatin from 293 cells cotransfected with Six1 expression plasmid and the Fgf8+25000, SIXmt1 or SIXmt2 reporter transgene found that SIXmt1 only decreased Six1-binding in 293 cells and weakened enhancer activity in vivo (n=5/5 transgenic lines), whereas SIXmt2 disrupted Six1-binding in 293 cells and abolished transcriptional activity in vivo (n=7/7 transgenic lines). This demonstrates that Six1-binding is necessary for inner hair cell-specific enhancer activity.

Dusp6 is a downstream effector of Fgfr signaling and inactivation of Dusp6 causes hearing loss. In vivo examination of Dusp6+2260 with strong H3K27ac-deposition revealed activity in the otocyst, cochlear inner-pillar cells and the spiral ganglion (n=8/8 transgenic lines), recapitulating the pattern of Dusp6 expression. This region contains an RFX-motif adjacent to the SIX-motif and ChIP-qPCR on E14.5-15.5 cochleae confirmed stronger enrichment by Rfx1 than Rfx3. A 3-bp mutation of the SIX-motif (SIXmt1) reduced Six1-binding (FIG. 5E,F) and weakened enhancer activity (n=3/3 transgenic lines). However, a 4-bp mutation of SIX alone (SIXmt2), which completely disrupted Six1-binding, or mutation of both RFX:SIX motifs abolished enhancer activity in the sensory epithelium, while spiral ganglion activity remained unperturbed (n=5/6 transgenic lines). Although the RFX-motif is non-redundant for enhancer activity in vivo, co-binding with Rfx1/3-binding may affect Six1-DNA binding affinity.

In contrast to the presence of Pbx1 expression in Six1-deficient cochlea, Dusp6 expression was almost completely lost in Six1-deficient cochlear sensory epithelium with residual expression in the apical end. This indicates that Dusp6 expression in vivo requires Six1 activity. Collectively, these data indicate that Six1 directly regulates inner-pillar-cell-specific Dusp6 expression by binding to the intronic Dusp6+2200 enhancer.

Inactivation of Six in Differentiating Hair Cells Disrupts Both Hair-Bundle Structural Polarity and Planar Cell Polarity (PCP)

To bridge the ChIP-seq data to cellular differentiation of the auditory sensory epithelium, Six1 was conditionally deleted in differentiating hair cells (tamoxifen at E14.5). On the apical surface, F-actin and anti-acetylated tubulin staining and scanning electron microscopy (SEM) revealed V-shaped stereocilia packed with actin filaments and a kinocilium centered next to the tallest stereocilia on each hair cell, which are uniformly aligned along the medial-lateral axis across the entire sensory epithelium (referred as PCP). The stereocilia and kinocilium are interconnected by distinct types of hair-bundle links to maintain the intrinsic structural polarity. SEM also revealed flatter inner hair cell bundles and V-shaped outer hair cell bundles at P0. The apical surface of Six1^{Cko/Cko} sensory epithelium displayed disrupted intrinsic polarity and PCP with a range of both structural deformation and misorientation.

The primary hair-bundle defects include flat bundle, multiple groups of stereocilia within the same cell (split), and very few stereocilia. The kinocilium was present on the lateral edge of the hair cell apical surface, indicating that kinocilia normally migrate from the center. However, the kinocilia were often found off-centered without connection to the stereocilia. Occasionally the kinocilium was found either centered within one group of stereocilia or absent. Overall, 78% of hair cells counted from the mid-basal cochlea displayed hair-bundle abnormalities.

The bundle orientation as a readout of hair cell PCP was also significantly disrupted in both inner hair cells and outer hair cells with outer hair cells more affected than inner hair cells. The angle measurements of misoriented bundles varied with some bundles in outer hair cells rotated up to 90-150°. Together, these observations demonstrate the importance of Six1 in both cell-intrinsic bundle morphogenesis and PCP during terminal differentiation.

Six1 Targets a Wide Range of Regulators Involved in Development of Primary Hair-Bundle and Orientation

Using UCSC liftOver, Six1 peaks were mapped to a total of 7495 genes in the human genome and found that 186 peaks mapped to 83 of the 152 deafness-associated genes collected in the Deafness Variation Database, which overlapped with over 2101 SNPs, of which >80% belong to unknown significance. Notably, mutations in many of these genes cause deafness due to hair-bundle abnormalities. These targets include myosin motors, actin binding, cytoskeletal, scaffolding, transmembrane, cell adhesion, multiple channel proteins, and G-protein signaling. Prominent Six1 targets of the PCP-signaling include Vangl1/Vangl2/Celsr1 and Ptk7. Immunostaining for Clic5, which is localized to hair-bundle(43), revealed significant reduction in Six1^{Cko/Cko}. As indicated by F-actin and anti-Vangl2 or -Celsr1 staining (FIG. 7D), while disorganization of cell-to-cell contacts was apparent in Six1^{Cko/Cko}, the expression of two key components of the Wnt/PCP signaling Vangl2/Celsr1, which are localized on the medial side of the hair cell membranes, was markedly reduced in the CKO sensory epithelium. qRT-PCR confirmed decreased expression of these targets in Six1^{Cko/Cko}.

In vivo examination of the intronic Vangl2+10200 CRE found activity in inner hair cells and surrounding supporting cells on the medial region of the sensory epithelium as well as in GER, but not in outer hair cells and their surrounding supporting cells on the lateral sensory epithelium. This CRE was also active in all vestibular hair cells and supporting cells. Thus, Vangl2 expression in medial versus lateral auditory sensory epithelium is mediated through distinct CREs. Collectively, these results provide insight into how Six1 regulates terminal differentiation through direct binding to CREs at key loci of both cell-intrinsic and intercellular planar polarity proteins to shape the auditory sensory epithelium.

### Example 2: Six1 Gene Therapy Restore Hearing Function In Vivo

To demonstrate the ability of Six1 gene therapy to restore hearing in mice, four groups of mice were used: (1) Experimental group Sox2^{CreER};R26-Six1-mCherry (treated with gentamicin and tamoxifen); (2) R26-Six1-mCherry (treated with gentamicin only, control group), (3) Sox2^{CreER} (treated with tamoxifen only, control group), and (4) R26-Six1-mCherry (treated with saline, control group).

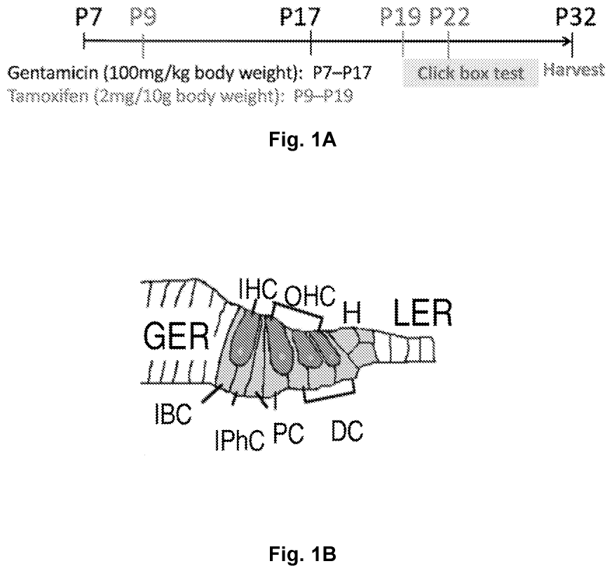

Transgenic mice were injected with gentamicin (i.p.) at 100 mg/kg body weight per day for 11 consecutive days from P7 to P17 to induce hair cell loss in the organ of the Corti (see FIG. 1A and Table 2). The animals also received an injection (i.p.) of 2 mg per 10 g body weight per 36 h of tamoxifen two days after gentamicin treatment as shown in Table 2. Then the animals were housed for another 13-15 days to allow hair cells to differentiate.

On days P 22, P25, P28, and P32, hearing was assessed using a click-box hearing test, in which a Preyer reflex or startle is elicited in response to auditory stimuli in hearing mice. Specifically, a custom built click box was held above the mouse to deliver a calibrated 20 kHz tone burst at an intensity of 90 dB sound pressure level (SPL) and the presence of an ear flick response (Preyer reflex) was recorded. The click box test identifies mice that have a severe or profound hearing impairment, and not those with mild to moderate deafness. After the click box test, inner ears were harvested at P32 and processed for immunostaining with anti-Myosin 7a antibody for detecting hair cells and anti-Sox2 antibody for detecting supporting cells. After immunostaining, cochleae were nuclear (DNA) counter stained with Hoechst. Whole mount organ of Corti were imaged using Nikon fluorescence microscope to show hair cells on the apical layer and supporting cells on the basal layer within the organ of Corti.

As shown in Table 2, control groups 3 (Sox2^{CreER} mice treated with tamoxifen) and 4 (R26_{Six1-mCherry} mice treated with saline) showed normal response to Preyer reflex test. Control group 2 (R26^{Six1-mCherry} mice treated with gentamicin to induce hair cell loss) showed no response. While the two animals in experimental group 1 (Sox2^{CreER}; R26^{Six1-mCherry} mice treated with gentamicin to induce hair cell loss and treated with tamoxifen to induce Six1 expression) did not show Preyer reflex response on P22, one animal began to show weak response to Preyer reflex test on P28, which became stronger on P32, while the second sample animal had weak response to Preyer reflex test on P25, which appeared progressively stronger from P28 to P32 (Table 2).

The organ of Corti comprises four layers of hair cells: one layer of inner hair cells and three layers of outer hair cells in the apical layer. Seven layers of supporting cells are found in the basal layer: one layer of inner border cells, one layer of inner phalangeal cells, one layer of inner pillar cells, one layer of outer pillar cells, and three layers of Deiters' cells. See FIG. 1B.

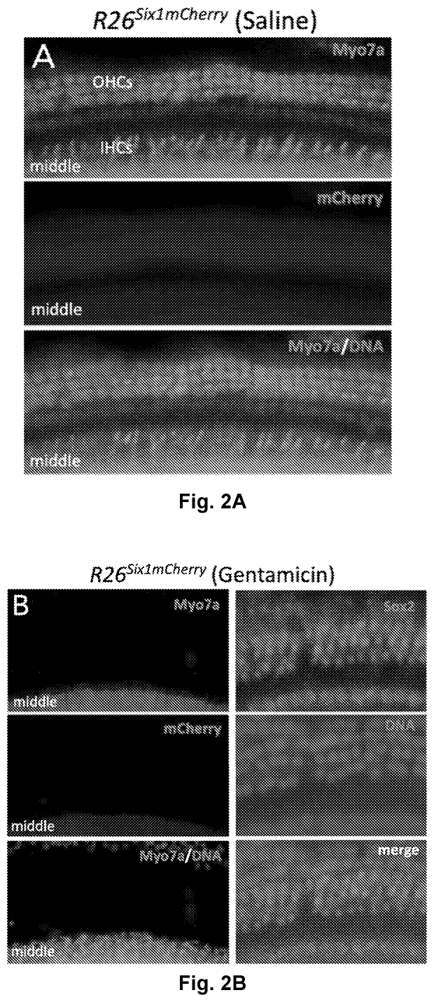

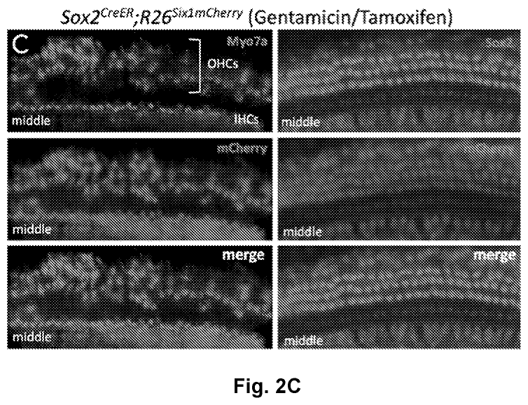

As expected, in all controls with normal response to Preyer reflex test (groups 3 and 4, Table 2), one row of inner and three rows of outer hair cells in the organ of Corti were present by anti-Myo7a staining (FIG. 2A). In control group 2, which had no response to Preyer reflex test), most of Myo7a⁺ hair cells were killed after treated with gentamicin for 11 days, especially in the basal and middle cochlear duct, but underlying Sox2⁺ supporting cells were present as revealed by immunostaining for Sox2 (FIG. 2B). In contrast, Myo7a⁺ and Cherry⁺ hair cells (indicating Six1 expression) were observed in the region of the outer hair cells in experimental group 1 treated with both gentamicin and tamoxifen (FIG. 2C). Anti-Sox2 immunostaining also revealed Sox2⁺ Cherry⁺ supporting cells in experimental group 1 (FIG. 2C), indicating that the new hair cells are unlikely regenerated through direct conversion of the supporting cells but instead through supporting cell proliferation and trans-differentiation.

In sum, this data shows that Six1 gene therapy can be successfully be used for hair cell regeneration and hearing restoration.

TABLE 2
Click box test. Responses to Preyer reflex test are (shown (−, +, ++,
and +++). +++ indicates a normal response. Gentamicin treatment served
to induce hair cells loss. Tamoxifen treatment to induce transgene
expression. Time points (P22, P25, P28, P32) are as in FIG. 1A.

| | | | Gentamicin | Tamoxifen | | | | |
|---|---|---|---|---|---|---|---|---|
| Group | Animal | Vectors | treatment | treatment | P22 | P25 | P28 | P32 |
| 1 | 1 | Sox2^{CreER}; | Yes | Yes | − | − | + | ++ |
| | 2 | R26^{Six1-mCherry} | | | − | + | ++ | ++ |
| 2 | 1 | R26^{Six1-mCherry} | Yes | No | − | − | − | − |
| | 2 | | | | − | − | − | − |
| 3 | 1 | Sox2^{CreER} | No | Yes | +++ | +++ | +++ | +++ |
| | 2 | | | | +++ | +++ | +++ | +++ |
| 4 | 1 | R26^{Six1-mCherry} | No | No | +++ | +++ | +++ | +++ |
| | 2 | | | | +++ | +++ | +++ | +++ |

### Example 3: Lentiviral Delivery for Regeneration of Hair Cells

Lentiviral vectors are generated for the expression of the following transgenes: (1) Six1 alone, (2) Eya1 and Six1, (3) Six1, Atoh1, and Pou4f3, and (4) Six1, Atoh1, Pou4f3, and Gfi11.

mESCs are infected as follows: 24-well plates are coated with 0.1% gelatin for 30 min, then washed with PBS. 6×10⁵ mESCs are plated with mESC media, and infected with lentivirus for 6 h. mESCs are collected and washed once with PBS. Cells are trypsinized with 0.05% Trypsin/EDTA, and neutralized with DMEM+10% FBS. Cells are centrifuged at 1000 rpm for 5 min. The cells are resuspended in mESC media without LIF, and plated into low-adhesion 6-well plate. Embryoid body (EB) will form within 24 h. The media are changed every two days.

1. A viral vector comprising one or more polynucleotides encoding one or more gene products, wherein the gene products are selected from the group consisting of Six1, Eya1, Atoh1, Pou4f3, and Gfi1.

2. The viral vector according to claim 1, wherein the gene product is Six1.

3. The viral vector according to claim 1, wherein the gene products are Six1 and Eya1.

4. The viral vector according to claim 1, wherein the gene products are Six1, Atoh1, and Pou4f3.

5. The viral vector according to claim 1, wherein the gene products are Six1, Atoh1, Pou4f3, and Gfi1.

6. The viral vector according to claim 1, wherein the viral vector is an adenoviral (AV) vector, adeno-associated virus (AAV) vector, retroviral vector, lentiviral vector, or Herpes simplex type 1 (HSV1) vector.

7. The viral vector according to claim 6, wherein the viral vector is an AV or AAV vector.

8. The viral vector according to claim 1, wherein the expression of the one or more gene products is inducible.

9. A pharmaceutical composition comprising one or more vectors according to claim 1 and pharmaceutically acceptable carrier.

10. A method of treating damage to or loss of cochlear hair cells in a subject in need thereof, the method comprising:
a. administering to a supporting cell in the organ of Corti in a subject in need thereof one or more vectors according to claim 1; or
b. administering to the subject a cell transduced with one or more vectors according to claim 1.

11. A method of generating cochlear hair cells in a subject in need thereof, the method comprising:
a. administering to a supporting cell in the organ of Corti in a subject in need thereof one or more vectors according to claim 1; or
b. administering to the subject a cell transduced with one or more vectors according to claim 1.

12. A method of increasing hearing function in a subject in need thereof, the method comprising:
a. administering to a supporting cell in the organ of Corti in a subject in need thereof one or more vectors according to claim 1; or
b. administering to the subject a cell transduced with one or more vectors according to claim 1.

13. The method according to claim 10, wherein the cell transduced with one or more vectors is a supporting cell.

14. The method according to claim 10, wherein the subject has hearing loss.

15. The method according to claim 14, wherein the subject has age-related hearing loss, hereditary hearing loss, noise-induced hearing loss, disease-associated hearing loss, or hearing loss resulting from trauma.

16. A viral vector for use in the treatment of hearing loss in a subject in need thereof, the viral vector comprising one or more polynucleotides encoding one or more gene products, wherein the gene products are selected from the group consisting of Six1, Eya1, Atoh1, Pou4f3, and Gfi1.

17. A cell for use in the treatment of hearing loss in a subject in need thereof, wherein the cell is transduced with one or more vectors comprising one or more polynucleotides encoding one or more gene products, wherein the gene products are selected from the group consisting of Six1, Eya1, Atoh1, Pou4f3, and Gfi1.

18. The method according to claim 11, wherein the cell transduced with one or more vectors is a supporting cell.

19. The method according to claim 11, wherein the subject has hearing loss.

20. The method according to claim 12, wherein the cell transduced with one or more vectors is a supporting cell.


SEQUENCE LISTING (5 sequences)

SEQ ID NO: 1 - protein, 283 aa, Homo sapiens
  Six1 (1)..(283)
MSMLPSFGFTQEQVACVCEVLQQGGNLERLGRFLWSLPACDHLHKNESVLKAKAVVAFHRGNFRELYKILESHQFSPHNHPKLQQLWLKAHYVEAEKLRGRPLGAVGKYRVRRKFPLPRTIWDGEETSYCFKEKSRGVLREWYAHNPYPSPREKRELAEATGLTTTQVSNWFKNRRQRDRAAEAKERENTENNNSSSNKQNQLSPLEGGKPLMSSSEEEFSPPQSPDQNSVLLLQSNMGHARSSNYSLPGLTASQPSHGLQAHQHQLQDSLLGPLTSSLVDLG

SEQ ID NO: 2 - protein, 591 aa, Homo sapiens
  Eya1 (1)..(591)
MEMQDLTSPHSRLSGSSESPSGPKLDSSHINSTSMTPNGTEVKTEPMSSSEIASTAADGSLDSFSGSALGSSSFSPRPAHPFSPPQIYPSKSYPHILPTPSSQTMAAYGQTQFTTGMQQATAYATYPQPGQPYGISSYGALWAGIKTESGLSQSQSPGQTGFLSYGTSFGTPQPGQAPYSYQMQGSSFTTSSGLYSGNNSLTNSSGFNSSQQDYPSYPGFGQGQYAQYYNSSPYPAHYMTSSNTSPTTPSTNATYQLQEPPSGVTSQAVTDPTAEYSTIHSPSTPIKETDSERLRRGSDGKSRGRGRRNNNPSPPPDSDLERVFIWDLDETIIVFHSLLTGSYANRYGRDPPTSVSLGLRMEEMIFNLADTHLFFNDLEECDQVHIDDVSSDDNGQDLSTYNFGTDGFPAAATSANLCLATGVRGGVDWMRKLAFRYRRVKEIYNTYKNNVGGLLGPAKREAWLQLRAEIEALTDSWLTLALKALSLIHSRTNCVNILVTTTQLIPALAKVLLYGLGIVFPIENIYSATKIGKESCFERIIQRFGRKVVYVVIGDGVEEEQGAKKHAMPFWRVSSHSDLMALHHALELEYL

SEQ ID NO: 3 - protein, 351 aa, Homo sapiens
  Atoh1 (1)..(351)
MSRLLHAEEWAEVKELGDHHRHPQPHHVPPLTPQPPATLQARDLPVYPAELSLLDSTDPRAWLTPTLQGLCTARAAQYLLHSPELGASEAAAPRDEADSQGELVRRSGCGGLSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPNVGEQPPPPTASCKNDHHHLRTASSYEGGAGASAVAGAQPAPGGGPRPTPPGPCRTRFSGPASSGGYSVQLDALHFPAFEDRALTAMMAQKDLSPSLPGGILQPVQEDNSKTSPRSHRSDGEFSPHSHYSDSDEAS

SEQ ID NO: 4 - protein, 338 aa, Homo sapiens
  Pou4f3 (1)..(338)
MMAMNAKQPFGMHPVLQEPKFSSLHSGSEAMRRVCLPAPQLQGNIFGSFDESLLARAEALAAVDIVSHGKNHPFKPDATYHTMSSVPCTSTSPTVPISHPAALTSHPHHAVHQGLEGDLLEHISPTLSVSGLGAPEHSVMPAQIHPHHLGAMGHLHQAMGMSHPHAVAPHSAMPACLSDVESDPRELEAFAERFKQRRIKLGVTQADVGAALANLKIPGVGSLSQSTICRFESLTLSHNNMIALKPVLQAWLEEAEAAYREKNSKPELFNGSERKRKRTSIAAPEKRSLEAYFAIQPRPSSEKIAAIAEKLDLKKNVVRVWFCNQRQKQKRMKYSAVH

SEQ ID NO: 5 - protein, 423 aa, Homo sapiens
  Gfi1 (1)..(423)
MPRSFLVKSKKAHSYHQPRSPGPDYSLRLETVPAPGRAEGGAVSAGESKMEPRERLSPDSQLTEAPDRASASPNSCEGSVCDPCSEFEDFWRPPSPSVSPASEKSLCRSLDEAQPYTLPFKPYAWSGLAGSDLRHLVQSYRQCSALERSAGLSLFCERGSEPGRPAARYGPEQAAGGAGAGQPGRCGVAGGATSAAGLGLYGDFAPAAAGLYERPSTAAGRLYQDHGHELHADKSVGVKVESELLCTRLLLGGGSYKCIKCSKVFSTPHGLEVHVRRSHSGTRPFACEMCGKTFGHAVSLEQHKAVHSQERSFDCKICGKSFKRSSTLSTHLLIHSDTRPYPCQYCGKRFHQKSDMKKHTFIHTGEKPHKCQVCGKAFSQSSNLITHSRKHTGFKPFGCDLCGKGFQRKVDLRRHRETQHGLK